(19) United States
(12) Patent Application Publication
(10) Pub. No.: US 2008/0233656 A1
(43) Pub. Date: Sep. 25, 2008

(54) Method For Detecting a Fuel Additive Component

(75) Inventors: Jorn Karl, Ludwigshafen (DE); Armin Bader, Frankenthal (DE); Benjamin Kaufman, Hopewell Junction, NY (US)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/997,179

(22) PCT Filed: Jul. 27, 2006
(86) PCT No.: PCT/EP2006/064745

§ 371 Date: Jan. 29, 2008

(30) Foreign Application Priority Data
Aug. 3, 2005 (DE) 10 2005 037 112.4

(51) Int. Cl. G01N 33/22; G01N 21/78

(52) U.S. Cl. 436/93; 436/164; 436/96

(57) ABSTRACT

A process for qualitatively or quantitatively detecting a fuel additive component which is part of an analyte comprising a fuel and/or further fuel additive components, by contacting the analyte with an indicator and determining the change, caused by the interaction between fuel additive component and indicator, in the color properties of the indicator in the analyte.

The present invention relates to a process for qualitatively or quantitatively detecting a fuel additive component, especially in diesel fuel or gasoline fuel. The present invention further provides for the use of this process for qualitatively or quantitatively detecting a fuel additive component in diesel fuel or gasoline fuel.

Gasoline fuel consists of a hydrocarbon mixture which may comprise, for example, additions of oxygen-containing organic components and additives for improving the properties. Such additives which are used in unleaded gasoline fuel are, for example, antioxidants, corrosion inhibitors, metal deactivators and detergents. The additives are used, inter alia, in order to prevent corrosion, deposits in the intake system, sludge formation and valve clogging in an internal combustion engine. The additive concentrations in gasoline fuel are typically in the range below 0.1% by weight. The additives are usually metered in the form of additive packages by the fuel manufacturer and admixed with the gasoline fuel when the tankers are filled in the refinery.

In the case of diesel fuel too, the addition of additives for the improvement of quality has become widely established. Additive packages with a total concentration below 0.1% by weight are usually added to the diesel fuel. The most commonly used additives for diesel fuel are flow improvers, lubricity improvers, ignition improvers, detergents, corrosion inhibitors and antifoams.

The analytical detection of fuel additive components in the additive packages and especially in the fuel itself has to date been problematic. Owing to the small dosage of the packages in the fuel (typically from 150 to 600 mg/kg), the concentration of the individual additive components is only a few ppm. Moreover, the fuel, as a complex matrix of chemical compounds, complicates the analysis. To date, the only reliable detection for fuel additive components described in the prior art has been the mass spectroscopy analysis process to this applicant according to DE-A 102 46 210 (1). However, this process is costly and inconvenient in its performance and apparatus.

It was therefore an object of the present invention to provide a detection process performable in a simple manner for a fuel additive component, which can especially also be performed “on site”, i.e. in the refinery or at the filling station.

It has now been found that, surprisingly, the change, determinable readily especially by photometric means, in the color properties of an indicator which is contacted with the fuel additive component in an analyte and interacts with it is the suitable method for this process.

Accordingly, a process has been found for qualitatively or quantitatively detecting a fuel additive component which is part of an analyte comprising a fuel and/or further fuel additive components, which comprises contacting the analyte with an indicator and determining the change, caused by the interaction between fuel additive component and indicator, in the color properties of the indicator in the analyte.

The process according to the invention can be employed qualitatively and quantitatively, i.e. it can be used to detect whether a certain fuel additive component is present and in what amount it is present. The detection can be performed especially in the fuel itself, but also in the parent additive packages. The analyte, i.e. the sample to be determined, is thus the fuel or the fuel additive package which can be diluted if required with suitable inert solvents to perform the determination.

Useful indicators include in principle all indicators which change their color properties on contacting and interaction with the fuel additive component, whether they be classifiable in the region of visible light or in the region of fluorescence or chemiluminescence behavior.

In a preferred embodiment, an acid-base indicator (also known as a pH indicator or neutralization indicator) is used. The parent acids or bases of these indicators exhibit a color change which typically occurs in the visible region when they are protolyzed or deprotolyzed. Typical examples of acid-base indicators are cresol red, metanil yellow, thymol blue, m-cresol purple, tropaeolin OO, 2,6-dinitrophenol, benzyl orange, 2,4-dinitrophenol, benzopurpurin 4 B, dimethyl yellow, congo red, bromophenol blue, bromochlorophenol blue, methylorange, α-naphthyl red, bromocresol green, 2,5-dinitrophenol, mixed indicator 5, methyl red, ethyl red, chlorophenol red, carminic acid, alizarin red S, 2-nitrophenol, litmus, bromocresol purple, bromphenol red, 4-nitrophenol, alizarin, bromothymol blue, bromoxylenol blue, brazilin, nitrazine yellow, hematoxylin, phenol red, 3-nitrophenol, neutral red, cresol red, m-cresol purple, brilliant yellow, orange I, α-naphtholphthalein, thymol blue, p-xylenol blue, o-cresolphthalein, phenolphthalein, α-naphtholbenzein, thymolphthalein, water blue, alizarin yellow 2 G, alizarin yellow R, nile blue A, α-naphthol violet, nitramine, tropaeolin OOO2, tropaeolin O, epsilon blue and acid fuchsin.

The color change in acid-base indicators may, which is also true of other indicators, be a one-color or two-color change. It should be sharp and recognizable. When this is not the case, suitable individual indicators are mixed to give mixed indicators.

Acid-base indicators used with particular preference are bromocresol green, α-naphthyl red, 2,5-dinitrophenol, mixed indicator 5 or methyl red.

The acid-base indicator utilizes especially basic functionalities in the fuel additive component to obtain the color change.

Further indicators useable in principle for the process according to the invention are adsorption indicators, for example fluorescein or eosin, fluorescence indicators, for example fluorescein, eosin, benzoflavin, phloxin, chromotropic acid, methylumbelliferon, benzoquinoline, morine, naphthols, naphthionic acid, quinine, coumarin or acridin, chemiluminescence indicators, for example lucigenin, redox indicators, for example neutral red, safranin or methylene blue, and metal indicators (metallochromic indicators).

For a qualitative determination of a fuel additive component, it is observed whether a specific color change occurs when indicator and analyte are combined, for example, in the case of bromocresol green, from yellow (acidic range below pH 3.8) to blue (alkaline range above pH 5.4). The occurrence of the color change is the proof of the presence of the fuel additive component being sought. To ensure that the color change has not been triggered by other influences, a blank experiment is advisable with an analyte to which has been added a test amount of the fuel additive component to be detected, and/or an analyte which comprises unadditized fuel.

For a quantitative detection of a fuel additive component, the intensity of the color is determined on completion of the change in the color properties in the analyte, i.e. on completion of color change. In a preferred embodiment, a suitable method for this purpose is photometric determination, normally with use of commercial photometers. The measurement is generally carried out in such a way that a certain amount of analyte is admixed with a fixed amount of the indicator and mixed through (for example by shaking), and the sample is analyzed with light of a certain wavelength in a sample vessel (cuvette). The measured absorbance in correlation to a blank sample and to a calibration curve constructed with different amounts of the fuel additive component to be determined gives rise to the amount of fuel additive component in the analyte. When bromocresol green is used as the acid-base indicator, it is advisable, for example, to determine the absorbance photometrically at a wavelength of 620 nm.

In a preferred embodiment, the analyte used is diesel fuel or gasoline fuel which, in addition to the fuel additive component to be detected, may comprise further fuel additive components, i.e. the determination is performed directly on commercially available diesel or gasoline fuel supplied by the refineries.

Gasoline fuel for operating internal combustion engines (gasoline engines) in motor vehicles are typically crude oil raffinates which generally have a boiling range of from 70 to 180° C. They are typically C₅-C₁₂ hydrocarbon mixtures of alkanes, alkenes, cycloalkanes, cycloalkenes and aromatics in changing composition. Gasoline fuel is preferably used in unleaded form.

The process according to the invention for detecting a fuel additive component can in principle also be performed in kerosene as the analyte. Kerosene in relatively high-boiling gasoline quality (boiling range from about 180 to 270° C.) is used especially in the aviation sector.

Diesel fuels (middle distillate fuels) are typically crude oil raffinates which generally have a boiling range of from 100 to 400° C. These are usually distillates having a 95% point up to 360° C. or even higher. They may also be “ultra low-sulfur diesel” or “city diesel”, characterized by a 95% point of, for example, not more than 345° C. and a sulfur content of not more than 0.005% by weight, or by a 95% point of, for example, 285° C. and a sulfur content of not more than 0.001% by weight. In addition to diesel fuels obtainable by refining, whose main constituents are relatively long-chain paraffins, suitable diesel fuels are those which are obtainable by coal gasification or gas liquefaction [gas-to-liquid (GTL) fuels]. Also suitable are mixtures of the aforementioned diesel fuels with renewable fuels such as biodiesel or bioethanol. At the present time, diesel fuels with low sulfur content are of particular interest, i.e. with a sulfur content of less than 0.05% by weight, preferably of less than 0.02% by weight, in particular of less than 0.005% by weight and especially of less than 0.001% by weight of sulfur. Diesel fuels may also comprise water, for example in an amount up to 20% by weight, for example in the form of diesel-water microemulsions or as so-called white diesel.

In a preferred embodiment, the process according to the invention is employed for the detection of a polar fuel additive component with detergent action which is typically found as an additive in most fuel types, especially in diesel fuel and in gasoline fuel, and also in the parent additive compositions (additive packages) for diesel and gasoline fuel in particular. Suitable polar detergent additives having especially basic functionalities or polar function groups, which usually interact with the indicators in an acid-base interaction, are listed below.

The additive compositions and fuels mentioned may additionally comprise different further fuel additive components such as demulsifiers, carrier oils, solvents and diluents, corrosion inhibitors, antioxidants, metal deactivators, antistats, markers, flow improvers, lubricity improvers, ignition improvers and antifoams. Suitable further fuel additives are likewise listed below.

### A) Detergent Additives

Detergent additives (detergents) typically refer to deposition inhibitors for gasoline fuel and diesel fuel. The detergent additives are preferably amphiphilic substances which have at least one hydrophobic hydrocarbyl radical having a number-average molecular weight (Mn) of from 85 to 20 000 and at least one polar moiety selected from:
(a) mono- or polyamino groups having up to 6 nitrogen atoms, of which at least one nitrogen atom has basic properties;
(b) nitro groups, if appropriate in combination with hydroxyl groups;
(c) hydroxyl groups in combination with mono- or polyamino groups, in which at least one nitrogen atom has basic properties;
(d) carboxyl groups or their alkali metal or their alkaline earth metal salts;
(e) sulfonic acid groups or their alkali metal or alkaline earth metal salts;
(f) polyoxy-C₂— to —C₄-alkylene groups which are terminated by hydroxyl groups, mono- or polyamino groups, in which at least one nitrogen atom has basic properties, or by carbamate groups;
(g) carboxylic ester groups;
(h) moieties derived from succinic anhydride and having hydroxyl and/or amino and/or amido and/or imido groups; and/or
(i) moieties obtained by Mannich reaction of substituted phenols with aldehydes and mono- or polyamines;

The hydrophobic hydrocarbyl radical in the above detergent additives, which ensures the adequate solubility in the fuel, has a number-average molecular weight (Mn) of from 85 to 20 000, especially from 113 to 10 000, in particular from 300 to 5000. Typical hydrophobic hydrocarbyl radicals, especially in conjunction with the polar moieties (a), (c), (h) and (i), include polypropenyl, polybutenyl and polyisobutenyl radical each having Mn=from 300 to 5000, especially from 500 to 2500, in particular from 700 to 2300.

Examples of the above groups of detergent additives include the following:

Additives comprising mono- or polyamino groups (a) are preferably polyalkenemono- or polyalkenepolyamines based on polypropene or conventional (i.e. having predominantly internal double bonds) polybutene or polyisobutene having Mn=from 300 to 5000. When polybutene or polyisobutene having predominantly internal double bonds (usually in the β- and γ-position) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be, for example, ammonia, monoamines or polyamines, such as dimethylaminopropylamine, ethylenediamine, diethylenetriamine, triethylenetetramine or tetraethylenepentamine. Corresponding additives based on poly(iso)butene are described in particular in EP-8 244 616; corresponding additives based on polypropene are described in particular in WO 94/24231.

Further preferred additives comprising monoamino groups (a) are the hydrogenation products of the reaction products of polyisobutenes having an average degree of polymerization P of from 5 to 100 with nitrogen oxides or mixtures of nitrogen oxides and oxygen, as described in particular in WO 97/03946.

Further preferred additives comprising monoamino groups (a) are the compounds obtainable from polyisobutene epoxides by reaction with amines and subsequent dehydration and reduction of the amino alcohols, as described in particular in DE-A 196 20 262.

Additives comprising nitro groups (b), if appropriate in combination with hydroxyl groups, are preferably reaction products of polyisobutenes having an average degree of polymerization P of from 5 to 100 or from 10 to 100 with nitrogen oxides or mixtures of nitrogen oxides and oxygen, as described in particular in WO 96/03367 and WO 96/03479. These reaction products are generally mixtures of pure nitropolyisobutenes (e.g. α,β-dinitropolyisobutene) and mixed hydroxynitropolyiso-butenes (e.g. α-nitro-β-hydroxypolyisobutene).

Additives comprising hydroxyl groups in combination with mono- or polyamino groups (c) are in particular reaction products of polyisobutene epoxides obtainable from polyisobutene having preferably predominantly terminal double bonds and Mn from 300 to 5000, with ammonia or mono- or polyamines, as described in particular in EP-A 476 485.

Additives comprising carboxyl groups or their alkali metal or alkaline earth metal salts (d) are preferably copolymers of C₂-C₄₀-olefins with maleic anhydride which have a total molar mass of from 500 to 20 000 and of whose carboxyl groups some or all have been converted to the alkali metal or alkaline earth metal salts and any remainder of the carboxyl groups has been reacted with alcohols or amines. Such additives are disclosed in particular by EP-A 307 815. Such additives serve mainly to prevent valve seat wear and can, as described in WO 87/01126, advantageously be used in combination with customary fuel detergents such as poly(iso)buteneamines or polyetheramines.

Additives comprising sulfonic acid groups or their alkali metal or alkaline earth metal salts (e) are preferably alkali metal or alkaline earth metal salts of an alkyl sulfosuccinate, as described in particular in EP-A 639 632. Such additives serve mainly to prevent valve seat wear and can be used advantageously in combination with customary fuel detergents such as poly(iso)buteneamines or polyetheramines.

Additives comprising polyoxy-C₂-C₄-alkylene moieties (f) are preferably polyethers or polyether amines which are obtainable by reaction of C₂- to C₆₀-alkanols, C₆- to C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group and, in the case of the polyether amines, by subsequent reductive amination with ammonia, monoamines or polyamines. Such products are described in particular in EP-A 310 875, EP-A 356 725, EP-A 700 985 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (g) are preferably esters of mono-, di- or tricarboxylic acids with long-chain alkanols or polyols, in particular those having a minimum viscosity of 2 mm²/s at 100° C., as described in particular in DE-A 38 38 918. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Such products also have carrier oil properties.

Additives comprising moieties derived from succinic anhydride and having hydroxyl and/or amino and/or amido and/or imido groups (h) are preferably corresponding derivatives of polyisobutenylsuccinic anhydride which are obtainable by reacting conventional or highly reactive polyisobutene having Mn=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. The moieties having hydroxyl and/or amino and/or amido and/or imido groups are, for example, carboxylic acid groups, acid amides, acid amides of di- or polyamines which, in addition to the amide function, also have free amine groups, succinic acid derivatives having an acid and an amide function, carboximides with monoamines, carboximides with di- or polyamines which, in addition to the imide function, also have free amine groups, and diimides which are formed by the reaction of di- or polyamines with two succinic acid derivatives. Such fuel additives are described in particular in U.S. Pat. No. 4,849,572.

Additives comprising moieties (i) obtained by Mannich reaction of substituted phenols with aldehydes and mono- or polyamines are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having Mn=from 300 to 5000. Such “polyisobutene-Mannich bases” are described in particular in EP-A-831 141.

For a more precise definition of the fuel additives detailed individually, reference is explicitly made here to the disclosures of the abovementioned prior art documents.

Particular preference is given in the process according to the invention to detergent additives from group (h). These are in particular polyisobutenyl-substituted succinimides, especially the imides with aliphatic polyamines. Such polyisobutenyl-substituted succinimides are used principally as a polar fuel additive component with detergent action in diesel fuel.

### B) Demulsifiers

Demulsifiers are substances which bring about the demixing of an emulsion. They may be either ionogenic or nonionogenic substances which are effective at the phase boundary. Accordingly, all surface-active substances are in principle suitable as demulsifiers. Particularly suitable demulsifiers are selected from anion-active compounds such as the alkali metal or alkaline earth metal salts of alkyl-substituted phenol- and naphthalenesulfonates and the alkali metal or alkaline earth metal salts of fatty acids, and also uncharged compounds such as alcohol alkoxylates, e.g. alcohol ethoxylates, phenol alkoxylates, e.g. tert-butylphenol ethoxylate or tert-pentylphenol ethoxylate, fatty acids, alkylphenols, condensation products of ethylene oxide (EO) and propylene oxide (PO), for example also in the form of EO/PO block copolymers, polyethyleneimines or else polysiloxanes.

The additive composition and the fuel may additionally be combined with further customary components and additives. Mention should be made here, for example, of carrier oils without marked detergent action, these being employed in particular in the case of use in gasoline fuels. However, they are occasionally also used in middle distillates.

### C) Carrier Oils

Carrier oils are usually used in combination with detergent additives and exert a solvent or washing function together with them. Carrier oils are generally high-boiling, viscous, thermally stable liquids which cover a hot metal surface and thus prevent the formation or deposition of contamination on the metal surface.

Suitable mineral carrier oils are the fractions obtained in crude oil processing, such as bright stock or base oils having viscosities, for example, from the SN 500-2000 class; and also aromatic hydrocarbons, paraffinic hydrocarbons and alkoxyalkanols. Likewise useful is a fraction which is obtained in the refining of mineral oil and is known as “hydrocrack oil” (vacuum distillate cut having a boiling range of from about 360 to 500° C., obtainable from natural mineral oil which has been catalytically hydrogenated under high pressure and isomerized and also deparaffinized). Likewise suitable are mixtures of abovementioned mineral carrier oils.

Examples of synthetic carrier oils which are useful in accordance with the invention are selected from: polyolefins (poly-alpha-olefins or poly(internal olefin)s), (poly)esters, (poly)alkoxylates, polyethers, aliphatic polyether amines, alkylphenol-started polyethers, alkylphenol-started polyether amines and carboxylic esters of long-chain alkanols.

Examples of suitable polyolefins are olefin polymers having Mn=from 400 to 1800, in particular based on polybutene or polyisobutene (hydrogenated or unhydrogenated).

Examples of suitable polyethers or polyetheramines are preferably compounds comprising polyoxy-C₂-C₄-alkylene moieties which are obtainable by reacting C₂-C₆₀-alkanols, C₆-C₃₀-alkanediols, mono- or di-C₂-C₃₀-alkylamines, C₁-C₃₀-alkylcyclo-hexanols or C₁-C₃₀-alkylphenols with from 1 to 30 mol of ethylene oxide and/or propylene oxide and/or butylene oxide per hydroxyl group or amino group, and, in the case of the polyether amines, by subsequent reductive amination with ammonia, monoamines or polyamines. Such products are described in particular in EP-A 310 875, EP-A 356 725, EP-A 700 985 and U.S. Pat. No. 4,877,416. For example, the polyether amines used may be poly-C₂-C₆-alkylene oxide amines or functional derivatives thereof. Typical examples thereof are tridecanol butoxylates or isotridecanol butoxylates, isononylphenol butoxylates and also polyisobutenol butoxylates and propoxylates, and also the corresponding reaction products with ammonia.

Examples of carboxylic esters of long-chain alkanols are in particular esters of mono-, di- or tricarboxylic acids with long-chain alkanols or polyols, as described in particular in DE-A 38 38 918. The mono-, di- or tricarboxylic acids used may be aliphatic or aromatic acids; suitable ester alcohols or polyols are in particular long-chain representatives having, for example, from 6 to 24 carbon atoms. Typical representatives of the esters are adipates, phthalates, isophthalates, terephthalates and trimellitates of isooctanol, isononanol, isodecanol and isotridecanol, for example di-(n- or isotridecyl) phthalate.

Further suitable carrier oil systems are described, for example, in DE-A 38 26 608, DE-A 41 42 241, DE-A 43 09 074, EP-A 452 328 and EP-A 548 617, which are explicitly incorporated herein by way of reference.

Examples of particularly suitable synthetic carrier oils are alcohol-started polyethers having from about 5 to 35, for example from about 5 to 30, C₃-C₆-alkylene oxide units, for example selected from propylene oxide, n-butylene oxide and isobutylene oxide units, or mixtures thereof. Nonlimiting examples of suitable starter alcohols are long-chain alkanols or phenols substituted by long-chain alkyl in which the long-chain alkyl radical is in particular a straight-chain or branched C₆-C₁₈-alkyl radical. Preferred examples include tridecanol and nonylphenol.

Further suitable synthetic carrier oils are alkoxylated alkylphenols, as described in DE-A 10 102 913.

### D) Further Coadditives

Further customary additives are additives which improve the cold properties of the fuel, for example nucleators, flow improvers, paraffin dispersants and mixtures thereof, for example ethylene-vinyl acetate copolymers; corrosion inhibitors, for example based on ammonium salts of organic carboxylic acids, said salts tending to form films, or on heterocyclic aromatics in the case of nonferrous metal corrosion protection; dehazers; antifoams, for example certain siloxane compounds; cetane number improvers (ignition improvers); combustion improvers; antioxidants or stabilizers, for example based on amines such as p-phenylenediamine, dicyclohexylamine or derivatives thereof or of phenols such as 2,4-di-tert-butylphenol or 3,5-di-tert-butyl-4-hydroxyphenylpropionic acid; antistats; metallocenes such as ferrocene; methylcyclopentadienylmanganese tricarbonyl; lubricity improvers, for example certain fatty acids, alkenylsuccinic esters, bis(hydroxyalkyl) fatty amines, hydroxyacetamides or castor oil; and also dyes (markers). Amines are also added if appropriate to lower the pH of the fuel.

Suitable solvents and diluents are, for example, aromatic and aliphatic hydrocarbons, for example C₅-C₁₀-alkanes such as pentane, hexane, heptane, octane, nonane, decane, their constitutional isomers and mixtures; petroleum ether, aromatics such as benzene, toluene, xylenes and Solvent Naphtha; alkanols having from 3 to 8 carbon atoms, e.g. propanol, isopropanol, n-butanol, sec-butanol, isobutanol and the like, in combination with hydrocarbon solvents; and alkoxyalkanols. Suitable diluents are, for example, also the fractions obtained in crude oil processing, such as kerosene, naphtha or brightstock. Diluents used with preference in the case of middle distillates, in particular in the case of diesel fuels and heating oils, are naphtha, kerosene, diesel fuels, aromatic hydrocarbons such as Solvent Naphtha heavy, Solvesso® or Shellsol®, and mixtures of these solvents and diluents.

When detergent additives, for example those having the polar moieties (a) to (i), are used in the fuel, especially in diesel fuel or gasoline fuel, they are added to the fuel typically in an amount of from 10 to 5000 ppm by weight, in particular from 50 to 1000 ppm by weight.

When demulsifiers are used, they are added to the fuel typically in an amount of from 0.1 to 100 ppm by weight, in particular from 0.2 to 10 ppm by weight.

The other components and additives mentioned are, if desired, added in amounts customary therefor.

The present invention further provides for the use of the process described for qualitatively or quantitatively detecting a fuel additive component in a diesel fuel or gasoline fuel comprising the fuel additive component with or without further fuel additive components.

The process according to the invention is a detection process which is simple to perform for a fuel additive component, especially a basic detergent additive, in a fuel additive package or in the fuel itself, and can also be performed on site, i.e. in the refinery or at the filling station, with simple analytical means. The detection process is substantially independent of the origin of the particular fuel type, i.e. the composition of the particular fuel has no influence on change, caused by the interaction between fuel additive component and indicator, in the color properties of the indicator in the analyte.

### EXAMPLE

### Quantitative Determination of a Polyisobutenyl-Substituted Succinimide Detergent Additive in Diesel Fuel

Samples of commercial unadditized diesel fuel from various refineries and refinery cuts were each additized in amounts close to those in practice with the same amounts in each case of a detergent additive based on the imide of polyisobutenylsuccinic anhydride (number-average molecular weight of the polyisobutenyl radical: approx. 1000) and tetraethylenepentamine which had been added in the form of a customary diesel performance package. From the absorbance values determined with the different dosages of the detergent additive in the individual diesel fuel samples (analytes) in a commercial photometer, a corresponding calibration curve was constructed for the range from 0 to 170 ppm by weight of detergent additive (based on active substance). The indicator added for the photometric determination was 1.0 ml of an ethanolic bromocresol green solution (13 mg of bromocresol green in 100 ml of ethanol, red-orange solution) per 10 ml of additized diesel fuel. The measurements were performed in a 1 ml cuvette at a wavelength of 620 nm on completion of the color change in the analyte from yellow to blue, which was triggered and completed by intensive shaking of the samples with the indicator solution in an analytical flask.

From the correlation of the constructed calibration curve, it was then possible to determine, with the analytical method outlined above, diesel fuel samples which comprised the abovementioned detergent additive in unknown amount in addition to further fuel additives, the amounts of this detergent additive quantitatively with a precision of ±10%.

1: A process for qualitatively or quantitatively detecting a polar fuel additive component with a detergent action which is part of a diesel fuel or a gasoline fuel as an analyte, the fuel, in addition to the polar fuel additive component with the detergent action to be detected, may comprise further fuel additive components, said process comprises contacting the analyte with an indicator and determining a change, caused by an interaction between the fuel additive component and the indicator, in color properties of the indicator in the analyte.

2: The process according to claim 1, wherein the indicator used is an acid-base indicator.

3: The process according to claim 2, wherein the acid-base-indicator used is bromocresol green, α-naphthyl red, 2,5-dinitrophenyl, mixed indicator 5 or methyl red.

4: The process for quantitatively detecting a fuel additive component according to claim 1, wherein the intensity of the color is determined photometrically on completion of the change in the color properties in the analyte.

5: The process according to claim 1, wherein the polar fuel additive component with the detergent action has moieties which are derived from succinic anhydride and have hydroxyl and/or amino and/or amido and/or imido groups.

6: The process according to claim 5, wherein the polar fuel additive components with the detergent action is polyisobutenyl-substituted succinimides.

7: The process according to claim 1, wherein the diesel fuel or the gasoline fuel comprises the polar fuel additive component as the analyte which is detected with or without further fuel additive components.
